(19) United States
(12) Patent Application Publication
(10) Pub. No.: US 2008/0294262 A1
(43) Pub. Date: Nov. 27, 2008

(54) Cages for Setting and Intersomatically Fusing Vertebrae

(76) Inventor: Jerome Levieux, Bellevue (FR)

Correspondence Address: YOUNG & THOMPSON, 209 Madison Street, Suite 500, ALEXANDRIA, VA, 22314, US

(21) Appl. No.: 12/095,924

(22) PCT Filed: Dec. 4, 2006
(86) PCT No.: PCT/FR2006/002644

§ 371 Date: Jun. 3, 2008

(30) Foreign Application Priority Data
Dec. 5, 2005 (FR) 0512308

(51) Int. Cl. A61F 2/44; A61B 17/70

(52) U.S. Cl. 623/17.16; 606/280

(57) ABSTRACT

A system for setting and intersomatically fusing vertebrae comprises at least one case and a plate perpendicularly adjacent to the main plane of said case, wherein said plate (1) and case (2) are provided with a hole (21)and a stud (12) insertable and releasable lockable into said hole 21, the plate (1) is removable by a simple traction and a disassembling device and a kit comprises a system for setting and intersomatically fusing vertebrae and the disassembling device.

The present invention relates to cages for setting and intersomatic fusion of vertebrae, and to its applications, and also to a disassembly device.

The systems for setting and intersomatic fusion of vertebrae are used in surgery of the cervical spine. These systems are based on the use of a prosthesis, called an intervertebral cage, for re-establishing the anatomical distance between two adjacent vertebrae when the intervertebral disc is weakened. The cage permits relative immobilization of these vertebrae through growth of a bone graft fitted in the cage, in the intervertebral space.

FR-A-2 851 457, 2 736 538, 2 816 201, 2 808 673 and 2 7 90 945, for example, describe systems for setting and intersomatic fusion of vertebrae, comprising at least one cage.

An improved type of implant is the cage/plate assembly described in the patent FR-A-2 747 034. This system comprises an open inner cage which receives spongy bone, and which is intended to be placed between two vertebrae and permits placement of a bone graft or of a material promoting intersomatic fusion, in order to induce the fusion of the two vertebrae concerned. The cage described in this system comprises, on its anterior face, an external element forming a plate that extends in a plane substantially perpendicular to the plane of introduction of the cage on either side of the latter, and having, at each of its ends, means for anchoring on at least two adjacent vertebrae that are to be joined to each other. This device ensures the positioning and non-mobility of the cage. The outer plate can be produced in one piece with the inner cage, or these two elements can be produced separately and joined subsequently by connecting means. These connecting means lead to a “locking” of the cage/plate assembly, which does not allow for easy disassembly by the surgeon. This is because the plate, when it is to be dismantled, has to be moved in translation parallel to the plane of the anterior face of the cage, and recent surgical techniques referred to as minimally invasive techniques do not leave space around said plate to permit such a translation.

It can happen that the permanent presence of a plate on the anterior part of the cervical vertebrae inconveniences the patient.

It would therefore be desirable to have a system for setting and intersomatic fusion of vertebrae which would allow the plate to be easily removed while leaving the cage in place, in particular without having to first move it in translation.

It would also be advantageous for the surgeon to be able to choose the site where the screws for fixing the plate are to be implanted, and, in the event of his failing to implant a screw, to be able to choose another site for re-implanting another screw.

It would therefore be desirable to have a system for setting and intersomatic fusion of vertebrae which would allow the surgeon to freely position the plate on the vertebral bodies.

After lengthy research, the applicant has developed a cage in which disassembly of the cage/plate assembly is made easier. Moreover, in certain embodiments, the plate is able to turn freely as long as the screws are not implanted in the vertebral bodies.

It is for this reason that the subject matter of the present application is a system for setting and intersomatic fusion of vertebrae, comprising at least one cage and one plate arranged perpendicular to the main plane of said cage, characterized in that said plate and said cage have on the one hand a hole and on the other hand a stud which can be inserted and reversibly blocked in said hole, said plate being able to be removed by simple traction.

A cage can have various shapes, in particular cylindrical, frustoconical or parallelepipedal, and is preferably made of titanium, PEEK (polyetheretherketone) or any biocompatible material. It comprises anterior and posterior and left and right faces that determine the intervertebral spacing, and also upper and lower open faces.

The plate preferably has a generally plane parallelepipedal or oblong shape. It is advantageously elongate and in particular made of titanium, stainless steel or any other biocompatible material.

To ensure its fixation on adjacent vertebrae, the plate comprises, generally toward each of its two ends, a hole that allows said plate to be fixed with screws implanted in the vertebral bodies.

According to one embodiment of the invention, the cage comprises the stud, while the plate comprises the hole. However, according to a preferred embodiment of the invention, the cage comprises the hole, while the plate comprises the stud.

When the cage comprises the hole and the plate comprises the stud, the hole will advantageously be provided on the anterior lateral face of the cage. The stud for its part will advantageously be provided between the holes present toward each of the ends of the plate. Conversely, when the cage comprises the stud and the plate comprises the hole, the stud will advantageously be provided on the anterior lateral face of the cage while the hole will advantageously be provided between the holes present toward each of the ends of the plate.

The stud can be inserted and reversibly blocked in said hole and permits rotation of the plate relative to the cage. It can correspond to a projection of parallelepipedal shape but is preferably cylindrical or inscribed within a cylinder. These latter options permit easier rotation of the plate relative to the cage.

The relative shapes and dimensions of the stud and of the hole will be such that the stud can be inserted in the hole and blocked in traction. For example, a cylindrical stud will be able to cooperate with a hole of cylindrical shape, but also with a cube or hexagon shape, for example.

In a preferred embodiment of the invention, the stud has a system for blocking said stud in the hole, in particular formed by means of a raised flange present at the end of the stud and preferably of cylindrical shape, or by means of pins present at the end of the stud, or by means of a preferably beveled collar to facilitate introduction of the stud into the hole, situated intermediately along the length of the stud. In the latter case, a groove will be provided in the hole for insertion of said collar. The groove will then have one of its walls inclined in order to permit withdrawal of the plate by traction.

According to one embodiment of the invention, the hole is milled at its inner end in order to permit withdrawal of the plate by traction when the flange is present at the end of the stud.

Under preferred conditions of implementation of the invention, the stud is hollow, especially a hollow cylinder, and has longitudinal slits that give it properties of elasticity. Thus, when the stud is introduced into the hole, the wall elements of the stud are able to close toward each other to permit introduction, and then to recover their original configuration in order to permit blocking upon completion of the introduction.

According to another embodiment of the invention, the stud is provided, on the plate and has, in its inner part, a fixing system that permits fastening of a disassembly device, as will be seen further below. The fixing system can comprise, for example, a groove and in particular a thread.

It is also possible to provide a cage and a plate that are provided with holes, and a joining piece which, for example, has the shape of a preferably cylindrical peg, designed for example like the stud.

The cage advantageously comprises, on at least one of the lateral faces, at least one aperture that allows it to deform during the movements that occur in the everyday life of an individual, so as to trams it to a graft, situated inside said cage, mechanical stresses stimulating the growth of osteoblasts.

The apertures situated in the left and right lateral faces are advantageously substantially symmetrical and can be in the form of horizontal slits alternating or stacked or oblique in the case of a plurality of slits, in the form of contiguous or spaced holes, of parallelepipedal shape or preferably circular shape, aligned or non-aligned, or in the form of holes connected by a longitudinal slit. These apertures have the dual function of permitting vascularization of the graft and of giving the cage the desired properties of deformability. In such a case, the cage is preferably made of PEEK.

The present application also relates to a disassembly device for an above system, comprising a shaft equipped with a handle, said shaft having at one end a device for fastening the plate or the stud, installed in a tube which at one end has a bridge equipped with two spaced legs for placing on either side of the plate and for bearing on the cage, said shaft being able to be displaced longitudinally in the tube.

According to one embodiment, the shaft is able to slide and has a lever at, its end remote from the end provided with a fastening device, which lever, when actuated, permits the longitudinal displacement of the shaft, the unblocking of the stud, and the withdrawal of the plate by simple traction.

According to another embodiment of the invention, the shaft is threaded at one end and cooperates with an abutment formed on the tube. By being screwed into the internal thread, of the stud and blocked in translation by the tube, the shaft allows a traction to be exerted for unblocking the stud and withdrawing the plate. The bridge can be internally threaded along the shaft in order to better guide the latter.

According to another embodiment of the invention, the plate is fastened by providing hooks at the end of the shaft in order to hook onto the plate.

Under preferred conditions of implementation of the invention, the end of the shaft remote from the handle is provided with threads, while the stud has an internal thread permitting cooperation, with the end of the shaft. In cooperation with the “legs” bearing on the cage, this system allows the stud to be unblocked and the plate to be withdrawn by simple traction.

The present application also relates to a kit that comprises a system and a disassembly device as above.

The present application also relates to a kit that includes a system and disassembly device as above and also the screws used for screwing the plate onto the vertebrae.

The system for setting and intersomatic fusion of vertebrae comprising a cage and a plate as described above, and its disassembly device, which are the subject matter of the present invention, have very advantageous properties and qualities.

This system permits in particular easier withdrawal of the plate while leaving the cage in place, without having to first move it in translation, for example. Moreover, being rotatable, it allows the surgeon to freely position the plate on the vertebral bodies.

These qualities are illustrated below in the experimental part. They justify the use of this system for setting and intersomatic fusion of vertebrae, and of its disassembly device described above, in surgery of the cervical spine.

The setting system according to the invention can also include two or more systems comprising a cage and a plate as described above, these being connected to each other by said plates, which may or may not overlap one another in pairs.

The preferred conditions of use of the systems for setting and intersomatic fusion of vertebrae and of the disassembly devices described above also apply to the other subject matters of the invention mentioned above, in particular to the kits comprising them.

The invention will be better understood by reference to the attached drawings, in which:

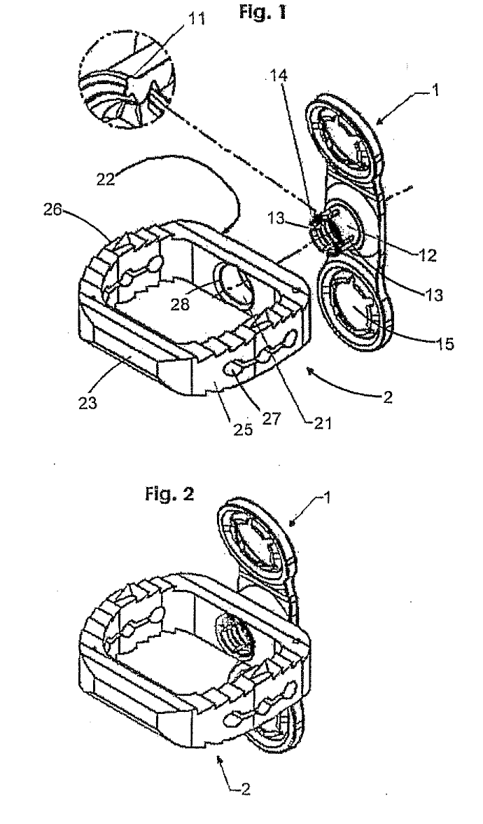

FIG. 1 shows a perspective view of a cage and a plate when disassembled, with the enlarged detail depicting the stud in partial cross section.

FIG. 2 shows a perspective view of the cage and plate when assembled.

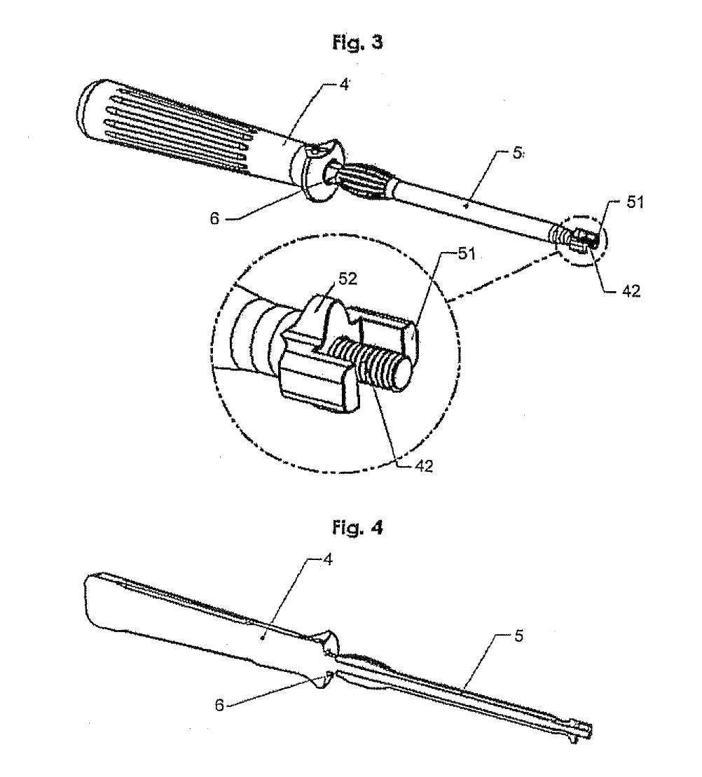

FIG. 3 shows a perspective view of a device for disassembly of the plate.

FIG. 4 shows a cross-sectional view of the device for disassembly of the plate.

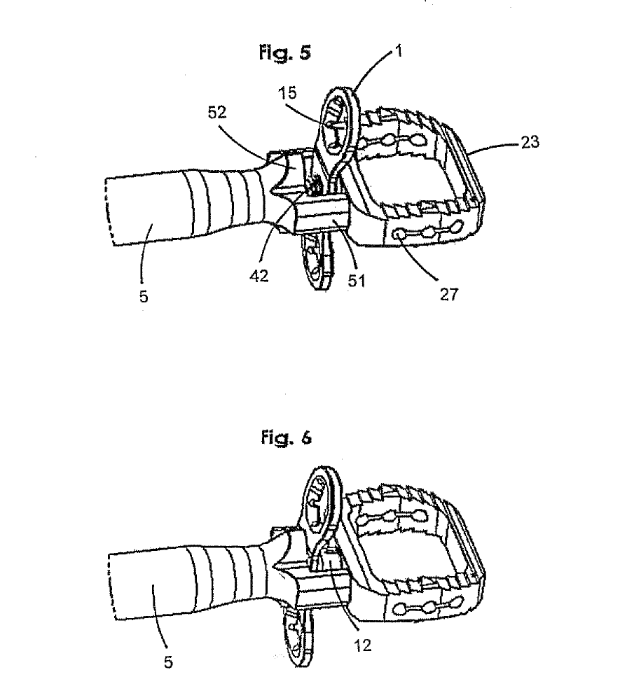

FIG. 5 shows a perspective view of the end of the device for disassembly of the plate, before traction is exerted, on the plate.

FIG. 6 shows a perspective view of the end of the device for disassembly of the plate, after traction has been exerted on the plate.

The directional expressions such as “anterior” and “posterior”, “top” and “bottom” or “upper” and “lower”, refer to the orientation of the cage when it is implanted in the vertebral column to be treated. Thus, in FIG. 1, the anterior lateral wall is arranged top right, and the posterior lateral wall is arranged bottom left.

As can be seen from FIG. 1, the system according to the invention comprises a plate 1 and a cage 2.

The cage 2 shown in FIG. 1 has in general a parallelepipedal shape and is symmetrical with respect to an antero-posterior plane passing through the main axis of the vertebral column. The cage 2 has an anterior lateral wall 22 and a posterior lateral wall 23 .

The anterior and posterior lateral walls 22, 23 are connected to each other by right 25 and left 26 lateral walls.

The cage 2 is completely open at the top and bottom and therefore has no upper or lower wall.

In the intervertebral cage illustrated, the four lateral walls rise perpendicularly with respect to a plane transverse to the axis of the column.

The right 25 and left 26 lateral walls are provided with elongate apertures 27 with an axis parallel to a plane transverse to the axis of the column. In FIG. 1, the shape of the aperture comprises three cylindrical holes connected via a diametral slit.

The cage 2 has, on the inside, a free volume delimited by the lateral walls and intended to be filled by a bone graft.

The anterior lateral wall 22 of the cage 2 has a cylindrical hole 21. The latter is milled 28 on its inside.

The plate 1, fitted perpendicular to the main plane of the cage 2, has a flattened and generally plane parallelepipedal shape, rounded at its ends.

It is integrally connected to a hollow cylindrical stud 12 which is provided with elongate notches 13 and with a bead 11 of slightly greater diameter than that of the hole 21 of the cage 2, but smaller that that of the milling 28. The four elongate notches 13 give a certain elasticity to the four cylinder portions thus formed, allowing the hollow stud 12 to be introduced into the hole 21 and then to be blocked therein by cooperation between the bead 11 and the milling 28.

The hollow stud 12 has, on its inside, a thread 14 designed to cooperate with the device described below for disassembly of the plate.

The plate 1 is anchored on adjacent vertebrae by way of screws (not shown) which pass through holes 14 formed at each of the ends of said plate 1, as can be seen in FIG. 1. These holes are arranged on the plate 1 on each side of the cylinder 12.

FIG. 2 shows the plate 1 and the cage 2 joined together. The plate 1 and the cage 2 can be pivoted relative to each other, thus permitting different positions of installation of the fixation screws.

FIG. 3 shows a disassembly device according to the invention. The tool is composed of a shaft 4 and of a tube 5.

At one end, the tube 5 has a bridge 52 provided with two legs 51 and, at the other end, it has a widened area that allows it to be gripped between the fingers, for example. The legs 51 are spaced apart by at least the width of the middle part of the plate 1.

The shaft 4 is threaded 42 at one end and has a handle at the other end, formed by a widening of the shaft 4. It extends through the tube 5 and comes into abutment at the end 6 of the tube 5 remote from the threaded, bridge 52.

The legs can thus be placed against the cage on either side of the plate. The threaded end 42 of the shaft 4 can be engaged in the internal thread of the hollow stud 12 and, by screwing through turning the handle, the plate 1 is withdrawn without its having to be displaced otherwise, especially in translation. The screwing thus produces a longitudinal displacement of the shaft 4 in the tube.

FIG. 4 shows the above disassembly device in cross section, illustrating the shaft 4, the tube 5, and the abutment 6 which is common to them.

FIG. 5 shows the device for disassembly of the plate, its legs 51 bearing on the cage 2 on either side of the plate 1. The threaded end 42 of the shaft 4 is screwed into the threaded internal part of the hollow stud 12 fitted on the plate 1.

FIG. 6 shows the disassembly device screwed onto the plate. By simple traction, the stud (12) has been unblocked and the plate 1 withdrawn, while the cage 2 remains in place.

The cage 2 shown has been made of PEEK. The plate 1 has been made of titanium.

1-11. (canceled)

12. A system for setting and intersomatic fusion of vertebrae, comprising at least one cage and one plate arranged perpendicular to the main plane of said cage, characterized in that said plate (1) and said cage (2) have a hole (21) and a stud (12) which can be inserted and reversibly blocked in said hole (21), permitting rotation of the plate (1) relative to the cage (2), said plate (1) being able to be removed by simple traction.

13. The system as claimed in claim 12, characterized in that the hole (21) is formed in the cage, and the stud (12) is fitted on the plate (1).

14. The system as claimed in claim 12, characterized in that the stud (12) is of cylindrical shape.

15. The system as claimed in claim 13, characterized in that the stud (12) is of cylindrical shape.

16. The system as claimed in claim 12, characterized in that the stud (12) is a hollow cylinder with longitudinal slits.

17. The system as claimed in claim 12, characterized in that the stud (12) has a system for blocking said stud (12) in the hole (21) provided in the plate (1).

18. The system as claimed in claim 12, characterized in that the stud (12) has a raised flange (11) at one end.

19. The system as claimed in claim 12, characterized in that the stud (12) has, in its inner part, a system for fixing of a disassembly device.

20. The system as claimed in claim 14, characterized in that the stud (12) has a thread in its inner part.

21. A disassembly device for a system for setting and intersomatic fusion of vertebrae comprising at least one cage and one plate arranged perpendicular to the main plane of said cage, in which said plate (1) and said cage (2) have a hole (21) and a stud (12) which can be inserted and reversibly blocked in said hole (21), permitting rotation of the plate (1) relative to the cage (2), said plate (1) being able to be removed by simple traction, characterized in that it comprises a shaft (4) equipped with a handle and has, at its end, a device (42) for fastening the plate (1) or the stud (12), installed in a tube (5) which at one end has two spaced legs (51) for placing on either side of the plate (1) and for bearing on the cage (2), said shaft (4) being able to be displaced longitudinally in the tube (5).

22. The disassembly device as claimed in claim 21, characterized in that the shaft (4) of the disassembly device is threaded (42) at its end remote from the handle.

23. A kit comprising a system for setting and intersomatic fusion of vertebrae as defined in claim 12, and a disassemly device comprising a shaft equipped with a handle and having, at its end, a device for fastening the plate (1) or the stud (12), installed in a tube (5) which at one end has two spaced legs (51) for placing on either side of the plate (1) and for bearing on the cage (2), said shaft (4) being able to be displaced longitudinally in the tube (5).
